SEQUENCE LISTING (6 sequences)

SEQ ID NO: 1 - DNA, 1632 bp, Acetinobacter sp.
atgtcacaaaaaatggattttgatgctatcgtgattggtggtggttttggcggactttatgcagtcaaaaaattaagagacgagctcgaacttaaggttcaggcttttgataaagccacggatgtcgcaggtacttggtactggaaccgttacccaggtgcattgacggatacagaaacccacctctactgctattcttgggataaagaattactacaatcgctagaaatcaagaaaaaatatgtgcaaggccctgatgtacgcaagtatttacagcaagtggctgaaaagcatgatttaaagaagagctatcaattcaataccgcggttcaatcggctcattacaacgaagcagatgccttgtgggaagtcaccactgaatatggtgataagtacacggcgcgtttcctcatcactgctttaggcttattgtctgcgcctaacttgccaaacatcaaaggcattaatcagtttaaaggtgagctgcatcataccagccgctggccagatgacgtaagttttgaaggtaaacgtgtcggcgtgattggtacgggttccaccggtgttcaggttattacggctgtggcacctctggctaaacacctcactgtcttccagcgttctgcacaatacagcgttccaattggcaatgatccactgtctgaagaagatgttaaaaagatcaaagacaattatgacaaaatttgggatggtgtatggaattcagcccttgcctttggcctgaatgaaagcacagtgccagcaatgagcgtatcagctgaagaacgcaaggcagtttttgaaaaggcatggcaaacaggtggcggtttccgtttcatgtttgaaactttcggtgatattgccaccaatatggaagccaatatcgaagcgcaaaatttcattaagggtaaaattgctgaaatcgtcaaagatccagccattgcacagaagcttatgccacaggatttgtatgcaaaacgtccgttgtgtgacagtggttactacaacacctttaaccgtgacaatgtccgtttagaagatgtgaaagccaatccgattgttgaaattaccgaaaacggtgtgaaactcgaaaatggcgatttcgttgaattagacatgctgatatgtgccacaggttttgatgccgtcgatggcaactatgtgcgcatggacattcaaggtaaaaacggcttggccatgaaagactactggaaagaaggtccgtcgagctatatgggtgtcaccgtaaataactatccaaacatgttcatggtgcttggaccgaatggcccgtttaccaacctgccgccatcaattgaatcacaggtggaatggatcagtgataccattcaatacacggttgaaaacaatgttgaatccattgaagcgacaaaagaagcggaagaacaatggactcaaacttgcgccaatattgcggaaatgaccttattccctaaagcgcaatcctggatttttggtgcgaatatcccgggcaagaaaaacacggtttacttctatctcggtggtttaaaagaatatcgcagtgcgctagccaactgcaaaaaccatgcctatgaaggttttgatattcaattacaacgttcagatatcaagcaacctgccaatgcctaa

SEQ ID NO: 2 - protein, 543 aa, Acetinobacter sp.
MSQKMDFDAIVIGGGFGGLYAVKKLRDELELKVQAFDKATDVAGTWYWNRYPGALTDTETHLYCYSWDKELLQSLEIKKKYVQGPDVRKYLQQVAEKHDLKKSYQFNTAVQSAHYNEADALWEVTTEYGDKYTARFLITALGLLSAPNLPNIKGINQFKGELHHTSRWPDDVSFEGKRVGVIGTGSTGVQVITAVAPLAKHLTVFQRSAQYSVPIGNDPLSEEDVKKIKDNYDKIWDGVWNSALAFGLNESTVPAMSVSAEERKAVFEKAWQTGGGFRFMFETFGDIATNMEANIEAQNFIKGKIAEIVKDPAIAQKLMPQDLYAKRPLCDSGYYNTFNRDNVRLEDVKANPIVEITENGVKLENGDFVELDMLICATGFDAVDGNYVRMDIQGKNGLAMKDYWKEGPSSYMGVTVNNYPNMFMVLGPNGPFTNLPPSIESQVEWISDTIQYTVENNVESIEATKEAEEQWTQTCANIAEMTLFPKAQSWIFGANIPGKKNTVYFYLGGLKEYRSALANCKNHAYEGFDIQLQRSDIKQPANA

SEQ ID NO: 3 - DNA, 1599 bp, Pseudomonas aeruginosa
gtggtagcaactaaagatttcgacgccatcgtcgttggcgcagggtttggcggtctgtatatgctgaaaaaactgcgcgacgagcagggtctcaatgttcgcgtcttcgacaaggcaggtgatgtgggcggaacgtggtactggaaccgctatccgggcgccctgtccgatactgaaacccatgtttactgctattcgtgggacaaagaactgttgcaggagatggatatcaccagtcggtacactacccagccgcaaatcctcaaatacctcgagaaagtggcggaccgccatgatctgcgcaaggatatccagttcaatactgggatcaccgcgatgcatttcaacgaaacgactaacctctgggaggtgcataccgacacgggtgagtcgtacactgccaagtttatcgtgactgcgcttggtctgctctccgccactaatattccaaaaattaaaggcctagagaccttccagagagagtgttaccacacgggtaactggccccaggatgttcagttcgagggaaaacgtgtgggcgtgatcggtacaggatccacaggtacccaggtgatcaccgccattgcgccacaggtcgagcatctcacagtgttccagcgttcgccgcaatacagcgtaccggtcggtaacggcccggtcagcagagagtatgtcgacaacatcaagaagaattacgacaaaatctgggagcaagtgaaaaactccatggtcgccttcggcttcgaggaaagcacggtgccggcgatgagtgtttctgatgaggaacgccaggcagtcttccagaaggcttgggaaaatggtggcggctttcgtttcatgtttgaaaccttctgcgacatcgccacagacgaacgtgccaacaaggccgcgcaggatttcatccggtccaagattgccgaaatcgtcaaggatccggaaactgctcgcaagctcacgccgaacgacctgtacgctaagcgccctctgtgcgacagcggttactacgcaacgtacaaccgcccgaacgtgagcctgctggacgtcaaggccaacccgatagcagagatcacccccaaaggggtgaaaaccgcagatggggtcgaacatgagttggatatgctgatctttgccaccggcttcgatgcggtggacggtaactacaccaagatcgatatccgaggccgtaaagggctggctatccaagaccactggaaggcaggcccgagcagctacctcggagttgcgaacgcaaattaccccaacatgttcatggtgttgggtcccaatgggcccttcaccaacctgccgccttcaatcgagacccaggtcgagtggatcagcgacctgatccaagacgtcaacactaagagtcttaagacagtggagcccacgcccgaggctgaggcattctggaccaagacttgccaggaaattgcaagcacgacgcttttcccgaaggcggagtcgtggatttttggtgccaatatcccgggcaagaccaacactgtatattttttcttggccggactcggtgcatatcggcaacaactcactgaagtacgaaagcaaggctaccaaggtttccagttcagtaaatga

SEQ ID NO: 4 - protein, 532 aa, Pseudomonas aeruginosa
MVATKDFDAIVVGAGFGGLYMLKKLRDEQGLNVRVFDKAGDVGGTWYWNRYPGALSDTETHVYCYSWDKELLQEMDITSRYTTQPQILKYLEKVADRHDLRKDIQFNTGITAMHFNETTNLWEVHTDTGESYTAKFIVTALGLLSATNIPKIKGLETFQRECYHTGNWPQDVQFEGKRVGVIGTGSTGTQVITAIAPQVEHLTVFQRSPQYSVPVGNGPVSREYVDNIKKNYDKIWEQVKNSMVAFGFEESTVPAMSVSDEERQAVFQKAWENGGGFRFMFETFCDIATDERANKAAQDFIRSKIAEIVKDPETARKLTPNDLYAKRPLCDSGYYATYNRPNVSLLDVKANPIAEITPKGVKTADGVEHELDMLIFATGFDAVDGNYTKIDIRGRKGLAIQDHWKAGPSSYLGVANANYPNMFMVLGPNGPFTNLPPSIETQVEWISDLIQDVNTKSLKTVEPTPEAEAFWTKTCQEIASTTLFPKAESWIFGANIPGKTNTVYFFLAGLGAYRQQLTEVRKQGYQGFQFSK

SEQ ID NO: 5 - DNA, 1225 bp, Pichia stipitis
atgcccatgtcttcagtcaaaatttctccattgaaggattctgaagcattccagtctatcaaagttggtaacaacactcttcaaaccaagattgtctatccaccaactactagatttagagctttagaagaccacactccttctgatttgcaattgcagtactatggcgacagatccactttcccaggtactttgcttatcactgaagctacttttgtctctcctcaagcctctggttatgaaggtgctgctccaggtatttggactgacaagcacgctaaagcatggaaggttattactgataaagttcatgccaacggttctttcgtttcaacccagttgatttttttgggaagggttgcagatccagctgttatgaagacccgtgggttgaatccagtttctgcctctgctacttatgaaagtgatgccgctaaagaagctgccgaagcagttggtaaccctgttagagctttgactacccaagaagtcaaggatcttgtttacgaggcttacaccaacgctgctcagaaggccatggatgctggtttcgactatattgaactccatgctgctcatggctaccttttagatcaatttttgcaaccatgcaccaatcaaagaactgatgaatacggtggatccattgagaacagagccaggttaattcttgagttgattgaccatttgtctaccattgtcggtgctgacaagattggtatcagaatctctccatgggctactttccaaaacatgaaggctcacaaggacactgttcacccattgactactttctcttacttggtccacgaattgcaacagagagctgacaagggtcaaggtattgcctacatttctgtcgttgagcctcgtgtaagtggtaacgtcgacgtctctgaagaagaccaagctggtgacaacgaatttgtctccaagatctggaagggtgttatcttgaaggcaggtaactactcctacgatgctccagagttcaagacattgaaggaagatatcgctgacaagcgtacattagttggcttctccagatacttcacctcgaatcctaacttggtttggaaattgcgtgatggaattgacttggtgccatacgacagaaacacgttctacagtgacaataactatggttacaataccttttctatggattccgaagaggttgataaagaattagaaatcaagagagttccttcggccattgaagctt

SEQ ID NO: 6 - protein, 408 aa, Pichia stipitis
MPMSSVKISPLKDSEAFQSIKVGNNTLQTKIVYPPTTRFRALEDHTPSDLQLQYYGDRSTFPGTLLITEATFVSPQASGYEGAAPGIWTDKHAKAWKVITDKVHANGSFVSTQLIFLGRVADPAVMKTRGLNPVSASATYESDAAKEAAEAVGNPVRALTTQEVKDLVYEAYTNAAQKAMDAGFDYIELHAAHGYLLDQFLQPCTNQRTDEYGGSIENRARLILELIDHLSTIVGADKIGIRISPWATFQNMKAHKDTVHPLTTFSYLVHELQQRADKGQGIAYISVVEPRVSGNVDVSEEDQAGDNEFVSKIWKGVILKAGNYSYDAPEFKTLKEDIADKRTLVGFSRYFTSNPNLVWKLRDGIDLVPYDRNTFYSDNNYGYNTFSMDSEEVDKELEIKRVPSAIEA

(19) United States
(12) Patent Application Publication
(10) Pub. No.: US 2021/0388403 A1
(43) Pub. Date: Dec. 16, 2021

(54) BIOCATALYTIC METHOD FOR PRODUCING 2H-HBO AND ß-SUBSTITUTED ANALOGUES FROM LGO USING A CYCLOHEXANONE MONOOXYGENASE

(71) Applicants: INSTITUT DES SCIENCES ET INDUSTRIES DU VIVANT ET DE L'ENVIRONNEMENT - AGROPARIS TECH, Paris Cedex 05 (FR); University of Florida Research Foundation, Inc., Gainesville, FL (US)

(72) Inventors: Florent Allais, Bouy (FR); Louis Mouterde, Reims (FR); John Dale Stewart, Gainesville, FL (US)

(21) Appl. No.: 17/291,821

(22) PCT Filed: Nov. 8, 2019
(86) PCT No.: PCT/FR2019/052677

§ 371 Date: May 6, 2021

(30) Foreign Application Priority Data
Nov. 8, 2018 (FR) 1860271

(51) Int. Cl. C12P 17/04

(52) U.S. Cl. CPC C12P 17/04; C12Y 114/13022

(57) ABSTRACT

An eco-compatible method is used to synthesize 2H-HBO optionally substituted at the β-position of the lactone function from LGO or a saturated form of LGO such as dihydrolevoglucosenone (2H-LGO) or LGO hydrate (OH-LGO) via a biocatalytic reaction using a cyclohexanone monooxygenase (CHMO).

### CROSS-REFERENCE TO RELATED APPLICATIONS

This application is a national phase entry under 35 U.S.C. § 371 of International Patent Application PCT/FR2019/052677, filed Nov. 8, 2019, designating the United States of America and published as International Patent Publication WO 2020/095008 A1 on May 14, 2020, which claims the benefit under Article 8 of the Patent Cooperation Treaty to French Patent Application Serial No. 1860271, filed Nov. 8, 2018.

### TECHNICAL FIELD

The present disclosure relates to the field of the preparation of 4-hydroxymethyl-γ-butyrolactone (2H-HBO) from levoglucosenone (LGO). More particularly, the present disclosure relates to an eco-compatible method for synthesis of 2H-HBO, optionally substituted at the β position of the lactone function, from LGO or a saturated form of LGO such as dihydrolevoglucosenone (2H-LGO) or the hydrate of LGO (OH-LGO) by means of biocatalytic reaction implementing a cyclohexanone monooxygenase (CHMO).

### BACKGROUND

In order to overcome the problems of dependency on fossil energies, various methods for preparing chemical compounds of interest have been developed, in recent decades, from biomass. Lignocellulosic biomass is one of the most exploited green sources of carbon compounds.

The levoglucosenone (LGO) is one of the products of most interest, which can be obtained in this way from biomass, in particular, by means of a technology of flash pyrolysis of cellulose. The LGO is commonly used as a starting product for the synthesis of various chemical compounds of interest, in particular, of 4-hydroxymethyl-γ-butenolide (HBO) and of 4-hydroxymethyl-α, β-butyrolactone (2H-HBO). The compounds constitute asymmetrical chemical intermediates (chirals) having significant added value; indeed, they are frequently used in the food-processing industry for preparing fragrances and aromas, or indeed in the pharmaceutical industry for the preparation of active ingredients of medication, taking advantage of the lactonic core thereof and of the chiral center thereof.

In the continuation of the framework of use of biosourced biochemical products obtained by green methods, it is desirable to develop industrial methods for transforming LGO into HBO and 2H-HBO or any other similar molecule, the ecological impact of which is as low as possible.

Conventional exploitation pathways of LGO into HBO and/or 2H-HBO are shown in FIG. 1. These pathways use metals, and also oxygenated water, two reagents of which the undesirable effects are well known, both for the environment and in an industrial exploitation context.

With the aim of proposing a more eco-compatible new preparation pathway for 2H-HBO, the inventors have sought to reduce, as far as possible, the use of organic solvents and toxic reagents.

The inventors' recent work has made it possible to propose a new pathway for hydrogenation of LGO into 2H-LGO, and of HBO into 2H-HBO, which does not use a metal, but rather an enzyme; alkene reductase OYE 2.6 (WO2018/183706).

However, the overall method of preparation of 2H-HBO, although without using metals, continues to consume oxygenated water (H₂O₂), a molecule whose use is difficult at the industrial level (danger during handling, pollution, etc.).

### BRIEF SUMMARY

A new method has been developed for preparing 2H-HBO from 2H-LGO using a natural enzyme, i.e., a cyclohexanone-monooxygenase (CHMO), as a replacement for oxygenated water.

The CHMO activity may, for example, be provided by an enzyme originating from Acinetobacter sp. or Pseudomonas aeruginosa, or any enzyme having the same activity.

The method has several advantages:
it is eco-compatible on account of the absence of organic solvent and metals;
it is industrially viable;
it does not exhibit risks of exothermicity and/or explosion, in contrast to the synthesis pathway using H2O2;
it can be implemented by means of biotransformation, in particular, using cells that express a cyclohexanone monooxygenase.

### BRIEF DESCRIPTION OF THE DRAWINGS

FIG. 1: Conventional exploitation pathways of LGO and/or 2H-LGO into 2H-HBO.

FIG. 2: Method for synthesis of 2H-HBO from LGO, and/or of 2H-LGO from a biocatylasis reaction using CHMO.

FIG. 3: “One-pot” synthesis method for 2H-HBO from LGO, by means of a biocatylasis reaction using CHMO and the alkene reductase OYE 2.6.

FIG. 4: Method for synthesis of 4-hydroxymethyl-γ-butyrolactone (2H-HBO), optionally substituted at the β position of the lactone function, from levoglucosenone (LGO) in saturated form.

### DETAILED DESCRIPTION

Proposed is a biocatalytic method for the preparation of 4-hydroxymethyl-γ-butyrolactone (2H-HBO), optionally substituted at the β position of the lactone function, from levoglucosenone (LGO) in saturated form, represented by formula (I),

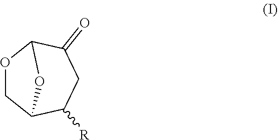

in which R represents an H or OH, NH₂, SH, linear, cyclic, or branched alkyl, OR₁ (where R₁ is a linear, cyclic, or branched alkyl, silyl, acyl, benzyl, benzoyle), NHR₁ (where R₁ is a linear, cyclic, or branched alkyl), NR₁R₂ (where R₁ and R₂ are a linear, cyclic, or branched alkyl), SR₁ (where R₁ is a linear, cyclic, or branched alkyl), or thioacetal group,

using a natural enzyme having CHMO activity.

Indeed, the inventors have previously demonstrated that an indispensable condition for the Baeyer-Villiger reaction of LGO using a CHMO is that the LGO is used in saturated form (for example, 2H-LGO, also referred to as CYRENE®).

“LGO in saturated form” means a levoglucosenone molecule of which the carbons in positions a and β of the cetone function are saturated. An example of a molecule saturated in positions a and β is 2H-LGO (or CYRENE®). In the general definition thereof, an LGO molecule in saturated form is represented by the formula (I) as defined above.

In a preferred embodiment, the LGO in saturated form is 2H-LGO and OH-LGO.

In another preferred embodiment, the LGO is in diastereoisomerically pure saturated form, represented by the formula (II),

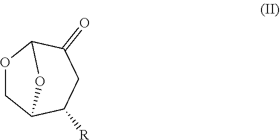

in which R represents an H or OH, NH₂, SH, linear, cyclic, or branched alkyl, OR₁ (where R₁ is a linear, cyclic, or branched alkyl, silyl, acyl, benzyl, benzoyle), NHR₁ (where R₁ is a linear, cyclic, or branched alkyl), NR₁R₂ (where R₁ and R₂ are a linear, cyclic, or branched alkyl), SR₁ (where R₁ is a linear, cyclic, or branched alkyl), or thioacetal group.

Thus, the object of the present disclosure relates, in particular, to a method for synthesis of 2H-HBO or OH—HBO from 2H-LGO or OH-LGO, respectively, comprising a biocatylasis reaction using a CHMO followed by an acid hydrolysis. More particularly, this method allows for the synthesis of 4-hydroxymethyl-γ-butyrolactone (2H-HBO), optionally substituted at the β position of the lactone function, such as (3 S,4R)-3-hydroxy-4-hydroxymethyl-γ-butyrolactone, (3S,4R)-3-methyl-4-hydroxymethyl-γ-butyrolactone, (3S,4R)-3-mercapto-4-hydroxymethyl-γ-butyrolactone, (3S,4R)-3-amino-4-hydroxymethyl-γ-butyrolactone, etc.

Any enzyme having CHMO activity can be implemented in this method.

“Enzyme having CHMO activity” or “CHMO” means, in particular, the CHMO originating from Acinetobacter sp. or CHMO of Pseudomonas aeruginosa, or any enzyme having the same activity, in particular variants thereof, “variant” means any enzyme having CHMO activity and having a sequence homology of at least 85% with one of the sequences SEQ ID NO:1 and SEQ ID NO:3, defined below. This homology is preferably at least 90%, indeed 95% or 98%.

Tables 1 and 2 show examples of such variants.

TABLE 1
Variants of CHMO of Acinetobacter sp. NCIMB9871

| | % homology |
|---|---|
| Organism | (BLAST) |
| Acinetobacter sp. NCIMB 9871 | 100.00% |
| Acinetobacter sp. SE19 | 99.82% |
| Acinetobacter baumannii | 99.63% |
| Acinetobacter junii | 97.97% |
| Acinetobacter sp. YT-02 | 97.42% |

TABLE 2
Variants of CHMO of Pseudomonas aeruginosa
strain Pa 1242

| | % homology |
|---|---|
| Organism | (BLAST) |
| Pseudomonas aeruginosa strain Pa1242 | 100% |
| Pseudomonas plecoglossicida strain | 99.81% |
| NyZ12 | |
| Pseudomonas panacis | 89.81% |
| Pseudomonas veronii | 90.00% |

The nucleotide sequence of CHMO originating from the strain NCIMB 9871 of Acinetobacter sp. is represented by the sequence SEQ ID NO:1; the corresponding protein sequence is represented by the sequence SEQ ID NO:2.

The nucleotide sequence of CHMO originating from the strain Pa1242 of Pseudomonas aeruginosa is represented by the sequence SEQ ID NO:3; the corresponding protein sequence is represented by the sequence SEQ ID NO:4.

Thus, in a preferred embodiment of the present disclosure, the cyclohexanone monooxygenase is an enzyme coded by the sequences SEQ ID NO:1 or SEQ ID NO:3, or a variant of one of the enzymes having a sequence homology of at least 85% to one of the sequences, provided that the CHMO activity is preserved.

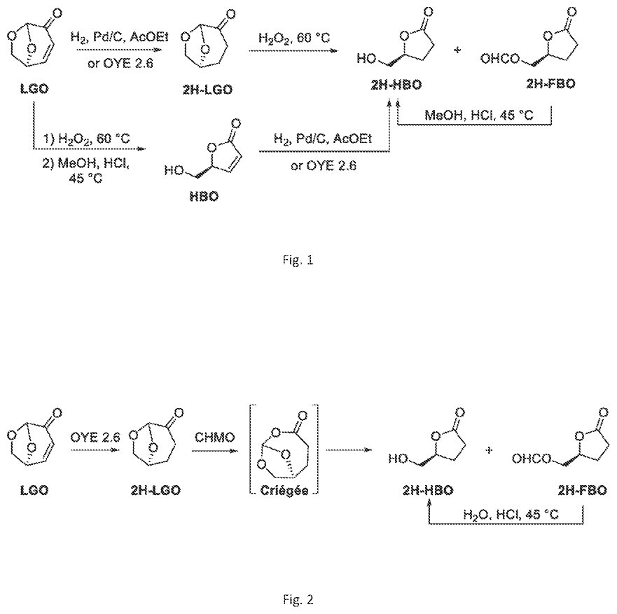

In a particular embodiment, the 2H-HBO synthesis reaction according to the present disclosure first comprises the conversion of the 2H-LGO into a relatively unstable intermediate, referred to as “Criegee,” which results in a second formulated intermediate thereof (2H-FBO). An acid hydrolysis then makes it possible to obtain 2H-HBO from 2H-FBO. A reaction of this kind is shown in its entirety in FIG. 2.

The same type of conversion involving the formation of an unstable intermediate is observed during the synthesis reaction of OH-HBO from OH-LGO, and more generally during the synthesis of 4-hydroxymethyl-γ-butyrolactone (2H-HBO), optionally substituted at the β position of the lactone function, from levoglucosenone (LGO) in saturated form.

The step of acid hydrolysis can be carried out by any method known to a person skilled in the art, for example, using hydrochloric acid, in particular, in a solution in methanol, acetic acid, sulfuric acid, a resin of the AMBERLYST® type, or indeed any acidic zeolite.

In another particular embodiment, the method according to the present disclosure further comprises a previous step of transformation of the LGO into a saturated molecule of formula (I), such as 2H-LGO. This transformation step can be carried out by means of heavy metals such as palladium, nickel or platinum, and dihydrogen. In a preferred embodiment, the step is implemented by a hydrogenation reaction that does not use heavy metals or dihydrogen (H₂). In the case where the method consists in the conversion of LGO into 2H-LGO, a reaction of this kind can be implemented using an alkene reductase enzyme such as OYE 2.6, as is set out in the application WO2018/183706, or any enzyme having the same activity, in particular variants thereof.

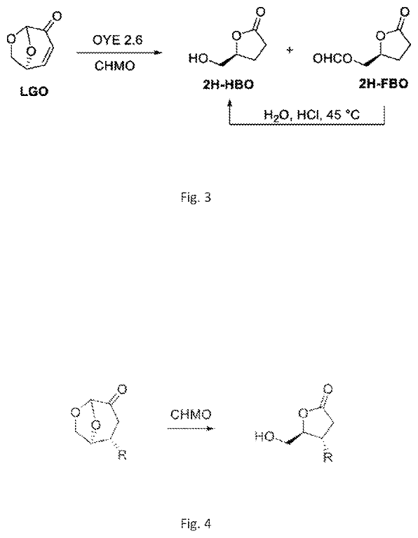

In a particular embodiment of the present disclosure, the transformation of the LGO into a saturated molecule is carried out within the same reaction medium (“one-pot” reaction), the reaction medium containing the catalysts or enzymes necessary for the transformation of the LGO into a saturated molecule of formula (I), and of the saturated molecule of formula (I) into a molecule of 4-hydroxymethyl-γ-butyrolactone (2H-HBO), optionally substituted at the β position of the lactone function, such as 2H-HBO. It is thus possible to obtain, in a simplified manner, 2H-HBO from LGO, in a single step. This reaction is shown in FIG. 3.

In a preferred embodiment of the one-pot method, the transformation of LGO into 2H-LGO is achieved by virtue of the action of an alkene reductase, for example, OYE 2.6.

The sequence of the alkene reductase OYE 2.6 is represented by the nucleotide sequence SEQ ID NO:5; the corresponding protein sequence is represented by the sequence SEQ ID NO:6.

In another preferred embodiment of the one-pot method, the transformation of 2H-LGO into 2H-HBO, or of OH-LGO into OH-HBO, is achieved by virtue of the action of a CHMO, in particular, the CHMO of Acinetobacter sp. or the CHMO of Pseudomonas aeruginosa or a variant of the enzymes.

In a particularly preferred embodiment of the one-pot method, the transformation of LGO into 2H-LGO is achieved by virtue of the action of an alkene reductase, and the transformation of 2H-LGO into 2H-HBO is achieved by virtue of the action of a CHMO.

A “one-pot” reaction of this kind can be achieved in a synthetic medium or in biotransformation, using whole cells. For the biotransformation, the cells used can be any type of cells suitable for this use, such as bacteria or animal or vegetable cells.

The various embodiments described above relate to the same general reaction, and the combination thereof is intended within the scope of this disclosure.

The present disclosure will be better understood upon reading the following examples, which are given by way of example and should under no circumstances be considered as limiting the scope of the present disclosure.

### EXAMPLES

### Example 1: Preparation of 2H-HBO from 2H-LGO in a Synthetic Medium

150 μl of purified CHMO was added to a solution containing 20 mM CYRENE®, 50 mM glucose, 0.5 mM NADP+, 0.25 mM FADH2, and 62.5 U glucose dehydrogenase (GDH), in a final volume of 25 ml, having 0.1 M phosphate buffer of pH 8.0. The solution was stirred magnetically (200 rpm) for a period of 12 hours, at 37° C.

The total consumption of CYRENE® was verified by thin-film chromatography. The analysis revealed that the CYRENE® was completely transformed into an intermediate that is neither 2H-FBO nor 2H-HBO (intermediate referred to as “Criegee”). The solution was subsequently evaporated under vacuum, and the raw material was analyzed by RMN, confirming the thin-film chromatography analyses.

An acid hydrolysis (HCl, 2 hours at 45° C.) makes it possible to convert this intermediate into 2H-FBO and then 2H-HBO.

A negative control was carried out, resuming the same conditions without the addition of CHMO. In these conditions, no transformation of CYRENE® was observed (thin-film chromatography), which confirms the action specificity of CHMO on the transformation of CYRENE®.

### Example 2: Biopreparation of 2H-HBO from 2H-LGO in a Cellular System in Bacteria Expressing CHMO of Acinetobacter sp

Escherichia coli BL21 (DE3) bacteria containing a plasmid bearing a gene resistant to Ampicillin and coding the enzyme CHMO of Acinetobacter sp. were placed in a culture in 1 ml LB medium containing ampicillin at a concentration of 100 μg/l and incubated at 37° C./200 rpm for a period of 12 hours. 500 μl of this solution were then transferred into 50 ml LB medium containing ampicillin at a concentration of 100 μg/1, incubated at 37° C. while stirring until a DO₆₀₀≈0.9 was achieved. The solution was then centrifugated at 4500 rpm at 4° C. for a period of 15 minutes. The supernatant was eliminated, and the cells were re-suspended in a minimum of 50 ml of medium M9. IPTG and CYRENE® were then added at final concentrations of 0.15 mM and 10-40 mM, respectively.

The total consumption of CYRENE® was verified by thin-film chromatography (maximum concentration of 5.2 g/l) (NB: the system can accept up to 10 g/l, but at such a concentration the conversion of CYRENE® is not complete). The analysis by thin-film chromatography has demonstrated that the CYRENE® is entirely transformed into an intermediate referred to as “Criegee,” which is different from 2H-FBO and from 2H-HBO, while at 10 g/l a mixture is obtained containing CYRENE®, the intermediate in question, 2H-FBO, and 2H-HBO. An acid hydrolysis (HCl, 2 hours at 45° C.) makes it possible to transform the intermediate, referred to as “Criegee” into 2H-FBO and then a molecule of interest 2H-HBO (validated by RMN analysis).

### Example 3: Biopreparation of 2H-HBO from 2H-LGO in a Cellular System in Bacteria Expressing CHMO of the Strain Pa1242 of Pseudomonas aeruginosa

The same methodology as in Example 2 was used to transform an Escherichia coli BL21 strain using a plasmid bearing a sequence originating from the strain Pa1242 of Pseudomonas aeruginosa. The sequence introduced was described as potentially coding for an enzyme having NAD(P)/FAD-dependent oxidoreductase activity, and thus capable of bearing a cyclohexanone monooxygenase activity.

It has been demonstrated, for the first time, that this sequence has the suspected activity. Indeed, the ability to convert CYRENE® into 2H-HBO has been tested and has thus demonstrated that it does indeed bear a cyclohexanone monooxygenase activity.

Total conversion is observed for CYRENE® concentrations of 1.25, 2.5 and 5 g/l, and partial conversion for concentrations of 10 and 20 g/l.

An RNM analysis has confirmed an increased purity, of 2H-HBO, of the order of 95%.

### Example 4: Biopreparation of OH-HBO from OH-LGO in a Cellular System in Bacteria Expressing CHMO

The experiments were carried out as described in Examples 2 and 3. Total conversion is observed for OH-LGO concentrations of 1 g/l. Moreover, the preparation of a single enantiomer is observed, as shown in FIG. 4.

1. A method for synthesis of 4-hydroxymethyl-γ-butyrolactone (2H-HBO), optionally substituted at the β position of the lactone function, from levoglucosenone (LGO) in saturated form, represented by formula (I), 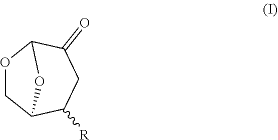
wherein R represents an H or OH, NH₂, SH, linear, cyclic or branched alkyl, OR₁ (where R₁ is a linear, cyclic or branched alkyl, silyl, acyl, benzyl, benzoyle), NHR₁ (where R₁ is a linear, cyclic or branched alkyl), NR₁R₂ (where R₁ and R₂ are a linear, cyclic or branched alkyl), SR₁ (where R₁ is a linear, cyclic or branched alkyl), or thioacetal group,
wherein the method comprises a biocatylasis reaction using a cyclohexanone monooxygenase followed by an acid hydrolyis.

2. The method of to claim 1, wherein the levoglucosenone (LGO) in saturated form is diastereoisomerically pure and is represented by formula (II), 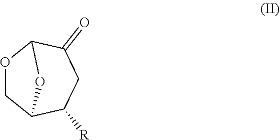
wherein R represents an H or OH, NH₂, SH, linear, cyclic or branched alkyl, OR₁ (where R₁ is a linear, cyclic or branched alkyl, silyl, acyl, benzyl, benzoyle), NHR₁ (where R₁ is a linear, cyclic or branched alkyl), NR₁R₂ (where R₁ and R₂ are a linear, cyclic or branched alkyl), SR₁ (where R₁ is a linear, cyclic or branched alkyl), or thioacetal group.

3. The method of claim 2, wherein the 4-hydroxymethyl-γ-butyrolactone (2H-HBO), optionally substituted at the β position of the lactone function, is 4-hydroxymethyl-γ-butyrolactone (2H-HBO) or the hydrate of 4-hydroxymethyl-γ-butenolide (OH-HBO).

4. The method of claim 3, wherein the levoglucosenone in saturated form is dihydrolevoglucosenone (2H-LGO) or the hydrate of LGO (OH-LGO).

5. The method of claim 4, further comprising a previous step of conversion of the LGO into a saturated molecule of formula (I) as defined in claim 1.

6. The method of claim 5, wherein the LGO is converted into 2H-LGO and the conversion of the LGO into 2H-HBO is achieved by a reaction using an alkene reductase.

7. A method for synthesizing 4-hydroxymethyl-γ-butyrolactone (2H-HBO) from levoglucosenone (LGO), comprising placing, into the same reactive medium, an enzyme that allows for a conversion of the LGO into 2H-LGO and an enzyme that allows for the conversion of 2H-LGO into a molecule of 4-hydroxymethyl-γ-butyrolactone (2H-HBO), the enzyme for conversion of 2H-LGO into 4-hydroxymethyl-γ-butyrolactone (2H-HBO) being a cyclohexanone monooxygenase.

8. The method of claim 7, wherein the enzyme allowing for conversion of LGO into 2H-LGO is an alkene reductase enzyme.

9. The method of claim 7, wherein the cyclohexanone monooxygenase enzyme originates from a strain of Acinetobacter sp. or a strain of Pseudomonas aeruginosa.

10. The method of claim 9, wherein the cyclohexanone monooxygenase is an enzyme coded by the sequences SEQ ID NO:1 or SEQ ID NO:3, or a variant of one of the enzymes having a sequence homology of at least 85% to one of the sequences.

11. The method of claim 8, wherein the alkene reductase is alkene reductase OYE 2.6 coded by the sequence SEQ ID NO:5 or one of the variants thereof having the same activity.

12. The method of claim 7, wherein the synthesis reaction of 4-hydroxymethyl-γ-butyrolactone (2H-HBO), optionally substituted at the f3 position of the lactone function, is carried out in a synthetic medium.

13. The method of claim 7, wherein the synthesis reaction of 4-hydroxymethyl-γ-butyrolactone (2H-HBO), optionally substituted at the β position of the lactone function, is carried out by biotransformation, using whole cells.

14. The method of claim 2, wherein the 4-hydroxymethyl-γ-butyrolactone (2H-HBO), optionally substituted at the β position of the lactone function, is 4-hydroxymethyl-γ-butyrolactone (2H-HBO) or the hydrate of 4-hydroxymethyl-γ-butenolide (OH-HBO).

15. The method of claim 3, wherein the levoglucosenone in saturated form is dihydrolevoglucosenone (2H-LGO) or the hydrate of LGO (OH-LGO).

16. The method of claim 4, further comprising a previous step of conversion of the LGO into a saturated molecule of formula (I) as defined in claim 1.

17. The method of claim 16, wherein the LGO is converted into 2H-LGO and the conversion of the LGO into 2H-HBO is achieved by a reaction using an alkene reductase.

18. The method of claim 1, wherein the cyclohexanone monooxygenase enzyme originates from a strain of Acinetobacter sp. or a strain of Pseudomonas aeruginosa.

19. The method of claim 6, wherein the alkene reductase is alkene reductase OYE 2.6 coded by the sequence SEQ ID NO:5 or one of the variants thereof having the same activity.

20. The method of claim 1, wherein the synthesis reaction of 4-hydroxymethyl-γ-butyrolactone (2H-HBO), optionally substituted at the β position of the lactone function, is carried out in a synthetic medium.
